(19) United States
(12) Patent Application Publication
(10) Pub. No.: US 2022/0079519 A1
(43) Pub. Date: Mar. 17, 2022

(54) Smart Wearable Device

(71) Applicant: Greg Jirik, Lonsdale, MN (US)

(72) Inventor: Greg Jirik, Lonsdale, MN (US)

(21) Appl. No.: 17/384,912

(22) Filed: Jul. 26, 2021

Related U.S. Application Data
(60) Provisional application No. 63/078,942, filed on Sep. 16, 2020.

(51) Int. Cl. A61B 5/00; A61B 5/01; A61B 5/0205; G08B 7/06; G04G 21/02; G01K 1/02

(52) U.S. Cl. CPC A61B 5/681; A61B 5/742; A61B 5/01; A61B 5/02438; G08B 7/06; G04G 21/025; G01K 1/028; A61B 5/02055

(57) ABSTRACT

A smart wearable device for detecting and displaying a skin temperature reading. The smart wearable device may be a temperature sensing wristwatch comprising a temperature sensor for measuring wearer's body temperature while the watch is worn. The temperature sensing wristwatch further comprises a plurality of indicating lights which illuminate in different colors to identify elevated body temperatures and normal body temperatures. The detected temperature is clearly displayed on a display screen of the device in a highly visible manner and creates an awareness of symptoms such as running fever among others in order to prevent and control the spread of diseases.

### CROSS-REFERENCE TO RELATED APPLICATION

The present application claims priority to, and the benefit of, U.S. Provisional Application No. 63/078,942, which was filed on Sep. 16, 2020 and is incorporated herein by reference in its entirety.

### FIELD OF THE INVENTION

The present invention relates generally to the field of smart wearable devices. More specifically, the present invention relates to a smart wristwatch configured to display the wearer's body temperature in a highly visible manner. Accordingly, this disclosure makes specific reference thereto the present invention. Nonetheless, it is to be appreciated that aspects of the present invention are also equally applicable to other like applications, devices and methods of manufacture.

### BACKGROUND OF THE INVENTION

By way of background, various germs, bacteria, viruses and other airborne pathogens are continuously present in the environment and can be dangerous. When such harmful pathogens are inhaled by individuals, it may cause serious illness and even death in critical cases. Additionally, a pandemic which is an epidemic of any disease that has spread across a large region, for instance multiple continents or worldwide, affecting a substantial number of individuals is also caused by exponential spread of virus, bacteria or pathogens causing the infectious disease. There have been a number of pandemics such as smallpox, tuberculosis, Plague, Spanish Flu and others. Current pandemics include HIV/AIDS and Coronavirus disease 2019 (COVID-19) which have been badly affecting the lives of people. Such pandemics have caused death of people across the world.

Various personal and community preventive measures are taken for the containment and mitigation of pandemics and other infectious diseases. For example, at the personal level, individuals are advised hand hygiene, sanitization, wearing face-masks, and self-quarantine. In critical scenarios at community level, social distancing such as closing schools, cancelling mass gatherings, avoiding crowds, and the like may be advised. However, personal preventive measures may not be effectively followed by all the individuals and social distancing is not always feasible. This can lead to a greater chance of spread of diseases when people get in contact with other infectious individuals.

People are often unaware of early illness-related symptoms, such as running a fever. In such cases people going out in public places with an elevated temperature can expose others to harmful illnesses. Infected individuals that are unaware of early symptoms such as fever may inadvertently spread the disease to others. People usually require temperature measurement devices such as thermometers to determine if they have a fever. People are required to carry the temperature measurement devices with them to be aware of the symptoms at any point of time. However, only the person measuring the temperature of the individual knows if there is a danger which would not be necessarily communicated to others. In such scenarios, if the individual with the elevated temperature goes out in public then other people are exposed to harmful illnesses.

Therefore, there exists a long felt need in the art for a smart device for measuring the body temperature of individuals. There is also a long felt need in the art for a smart device which is portable and can be easily worn by the individuals. Additionally, there is a long felt need in the art for a temperature measurement device which displays the temperature in order to create the awareness of the illness symptoms amongst others. Moreover, there is a long felt need in the art for a temperature measurement devices which indicates normal and elevated temperature levels to notify the wearer and others of the user's current condition. Further, there is a long felt need in the art for a smart wearable device which is convenient to use and can has longer battery life. Finally, there is a long felt need in the art for a device to detect early illness symptoms and creating an awareness amongst others to prevent and control the spread of diseases.

In this manner, the novel smart wearable device of the present invention accomplishes all of the forgoing objectives, and provides a relatively convenient and effective solution to determine illness symptoms such as running fever, and creating an awareness of the wearer's body temperature amongst others in order to prevent the spread of illness. The wristwatch temperature display of the present invention is also user friendly, inasmuch as the device can be easily worn by the wearer and notify the wearer and other individuals in case the wearer has an elevated body temperature.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a smart wearable device. The smart wearable device may be a temperature sensing smart watch. The smart wearable device comprises a case and a wristband attachable to the case. The case comprises an outward facing side, a skin facing side, and a perimeter. The case further comprises a pair of lugs oppositely located along the perimeter for engaging and connecting the case to the wrist band. The wrist band may be a strap comprising an adjusting element for tightening and loosening the wrist band. Alternatively, the wrist band may be a single stretchy strap.

The smart wearable device further comprises a display component and a temperature sensing component. The display component is integrated into the outward facing side of the case. The temperature sensing component is integrated into the skin facing side of the case. The display component comprises a digital display and a lighting element. The digital display comprises a plurality of health indicators. The digital display may further comprise a time and date indicator. The plurality of health indicators comprise a numerical temperature indicator.

The lighting component is a light strip or band that is integrated into the display component adjacent to the perimeter of the case. The display component is configured to visibly indicate a status of a user's skin temperature detected by the temperature sensing component. The numerical temperature indicator is configured to indicate a numerical temperature. The lighting component is configured to illuminate in a first color when the detected user's skin temperature is within a normal temperature range. The lighting component is further configured to illuminate in a second color when the detected user's skin temperature is outside of the normal temperature range. The wrist band may further comprise an illumination element. The illumination element of the wrist band may similarly be configured to indicate when the detected user's skin temperature is within or outside of a normal temperature range.

The smart wearable device further comprises a proximity sensing component. The proximity sensing component is integrated into the skin facing side of the case. The proximity sensing component is configured to activate the plurality of health sensing components when the smart wearable device is worn to conserve energy. The smart wearable device may further comprise an audible alert component. The audible alert component may be a speaker configured to provide an audible alert if a status of one of the plurality of health indicators is outside of a normal range.

The smart wearable device further comprises a user input. The user input may be a dial attached to the case or a touch element within the digital display for controlling components of the smart wearable device. The smart wearable device may further comprise a processor and a wireless communications module. The processor is configured to accept inputs from the user input. The wireless communications module is configured to communicate the measured health indicators with additional electronic platforms.

The smart wearable device may further comprise an oxygen saturation component. The oxygen saturation component is integrated into the skin facing side of the case and is configured to measure oxygen saturation through the user's skin. The smart wearable device may further comprise a heart rate sensing component. The pulse sensing component is integrated into the skin facing side of the case and is configured to measure a heart rate or the user. The plurality of health indicators may further comprise an oxygen saturation indicator configured to display an oxygen saturation status, and a pulse indicator to configured to display a heart rate status.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of a smart wearable device of the present invention in accordance with the disclosed architecture;

FIG. 2 illustrates a rear perspective view one potential embodiment of a smart wearable device of the present invention in accordance with the disclosed architecture;

FIG. 3 illustrates a front overhead perspective view of one potential embodiment of the smart wearable device of the present invention in accordance with the disclosed architecture;

FIG. 4 illustrates a rear overhead perspective view of one potential embodiment of the smart wearable device of the present invention in accordance with the disclosed architecture;

FIG. 5 illustrates a front overhead view of one potential embodiment of the smart wearable device of the present invention in accordance with the disclosed architecture;

FIG. 6 illustrates a rear overhead view of one potential embodiment one potential embodiment of the smart wearable device of the present invention in accordance with the disclosed architecture;

FIG. 7 illustrates a diagrammatic representation of exemplary components of one potential embodiment of the smart wearable device of the present invention in accordance with the disclosed architecture; and

FIG. 8 illustrates a perspective view of one potential embodiment of the smart wearable device of the present invention being worn by a user and displaying a normal body temperature in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a smart wearable device for measuring the body temperature of individuals. There is also a long felt need in the art for a smart device which is portable and can be easily worn by the individuals. Additionally, there is a long felt need in the art for a temperature measurement device which displays the temperature in order to create the awareness of the illness symptoms amongst others. Moreover, there is a long felt need in the art for a temperature measurement devices which indicates normal and elevated temperature levels to notify the wearer and others of the illness symptoms. Further, there is a long felt need in the art for a smart wearable device which is convenient to use and can has longer battery life. Finally, there is a long felt need in the art for a device to detect early illness symptoms and creating an awareness amongst others to prevent and control the spread of diseases.

The present invention, in one exemplary embodiment, is a temperature displaying wristwatch to display a wearer's body temperature. The device comprises a digital display, a LED strip circumscribing the digital display, a metallic case covering the digital display and the LED strip, a skin temperature sensor on rear surface of the case such that the skin temperature sensor touches a wearer's skin when the wristwatch is worn by the wearer, the digital display displays the sensed temperature in a digital form in Fahrenheit or Celsius scale and the LED strip illuminate in a specific color to indicate if the recorded temperature is normal body temperature or high body temperature. The display shuts off when the wristwatch is not worn by a wearer and the LED strip also does not illuminate.

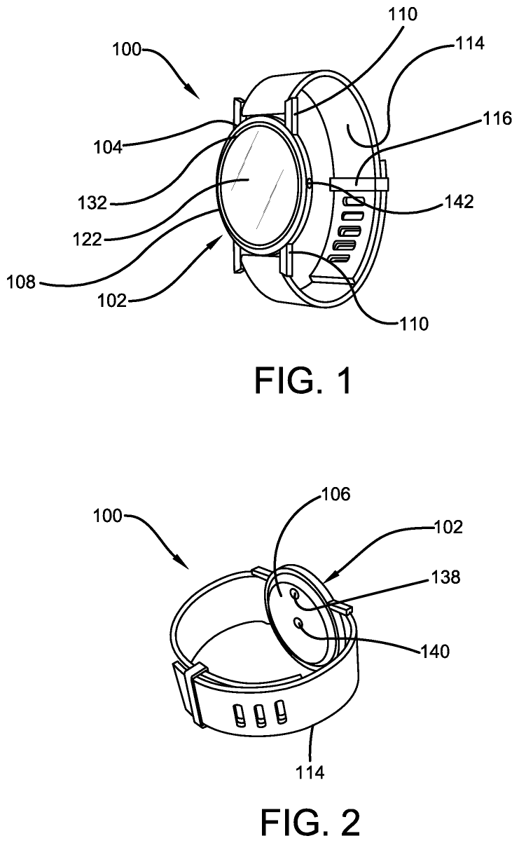

Referring initially to the drawings, FIGS. 1 and 2 illustrate a perspective view of a smart wearable device 100. The smart wearable device 100 may be a temperature sensing smart watch and is designed to display the wearer's body temperature in a highly visible manner. The smart wearable device 100 comprises a case 102 and a wristband 114 attachable to the case 102. The case 102 may be circular, oval, square, rectangular, or any other geometric shape as desired. The case 102 comprises an outward facing side 104, a skin facing side 106, and a perimeter 108. The case 102 further comprises a pair of lugs 110 oppositely located along the perimeter 108 for engaging and connecting the case 102 to the wrist band 114. The wrist band 114 may be a strap comprising an adjusting element 116 for tightening and loosening the wrist band 114. Alternatively, the wrist band 114 may be a single stretchy strap. The wrist band 114 may be constructed from any durable material such as silicone, rubber, plastic, leather, nylon, fibers, or the like.

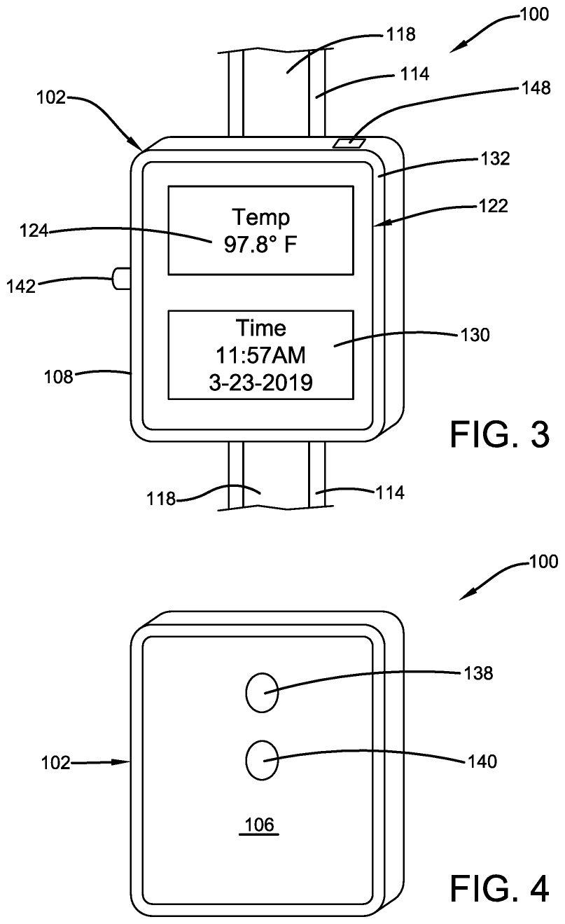

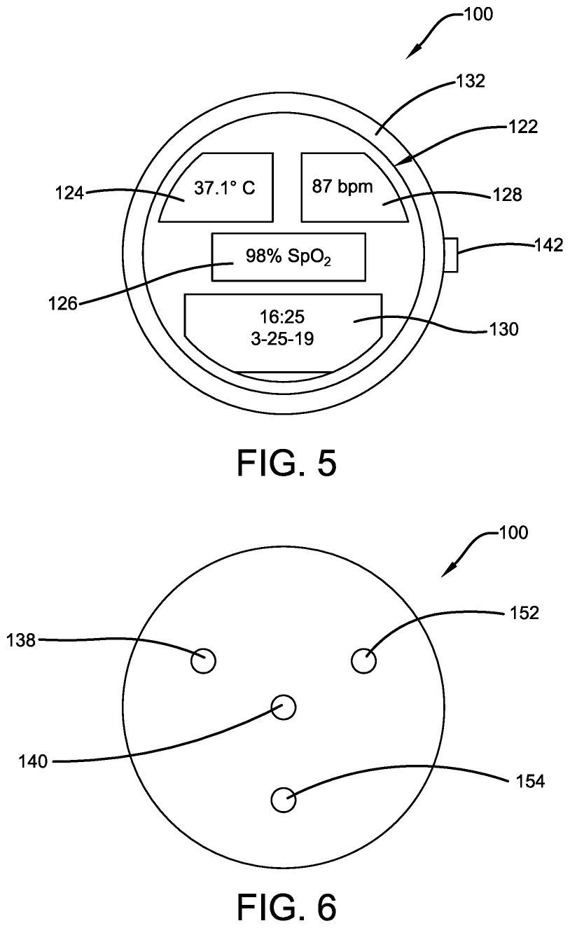

As illustrated in FIGS. 1-8, the smart wearable device 100 further comprises a display component 120 and a plurality of health indicating sensing components 138, 152, and 154. The plurality of health indicating sensing components 138, 152, and 154 comprise a temperature sensing component 138. The temperature sensing component 138 is configured to continuously measure the temperature of the wearer. The display component 120 is integrated into the outward facing side 104 of the case 102. The temperature sensing component 138 and other plurality of health indicating sensing components 152 and 154 are integrated into the skin facing side 106 of the case 102. The display component 120 comprises a digital display 122 and a lighting element 132.

The digital display 122 comprises a plurality of health indicators 124-128. The plurality of health indicators 124-128 comprise a numerical temperature indicator 124. The numerical temperature indicator 124 displays a temperature of the wearer in either degree Celsius or Fahrenheit. The digital display 122 may utilize ambient lighting to permit viewing of displayed information, may be backlit such that the displayed information may be viewed in the dark or other low-light environments, or may employ no light source at all. The digital display 122 may further comprise a time and date indicator 130. The time and date indicator 130 allow the smart wearable device 100 to also function as a watch. The digital display 122 may also feature a magnification lens (not shown) to enlarge a displayed temperature.

The lighting component 132c is a light strip or band that is integrated into the display component 120 adjacent to the perimeter 108 of the case 102. The lighting component 132 may be a LED strip surrounding the perimeter 108 of the case 102. The display component 120 is configured to visibly indicate a status of a user's skin temperature detected by the temperature sensing component 138. The numerical temperature indicator 124 is configured to display a numerical temperature.

The lighting component 132 is configured to illuminate in a first color when the detected user's skin temperature is within a normal temperature range. The lighting component 132 is further configured to illuminate in a second color when the detected user's skin temperature is outside of the normal temperature range. The wrist band 114 may further comprise an illumination element 118. The illumination element 118 of the wrist band 114 may similarly be configured to illuminate in a first color when the detected user's skin temperature is within a normal temperature range and illuminate in a second color when the detected user's skin temperature is outside of the normal temperature range.

The smart wearable device 100 further comprises a proximity sensing component 140. The proximity sensing component 140 is integrated into the skin facing side 106 of the case 102. The proximity sensor 140 detects the proximity of the smart wearable device 100 to a user skin and switches on the display component 120 when a wearer wears the smart wearable device 100. The proximity sensor 140 is used to determine whether the smart wearable device 100 is being worn by the wearer. The proximity sensor 140 can be an Infrared proximity sensor in one embodiment

The proximity sensing component 140 is also configured to activate the plurality of health sensing components 138, 152, and 154 when the smart wearable device 100 is worn to conserve energy. The smart wearable device 100 may further comprise an audible alert component 148. The audible alert component 148 may be a speaker integrated into the case 102 and configured to provide an audible alert if a status of one of the plurality of health indicators 124-128 is outside of a normal range.

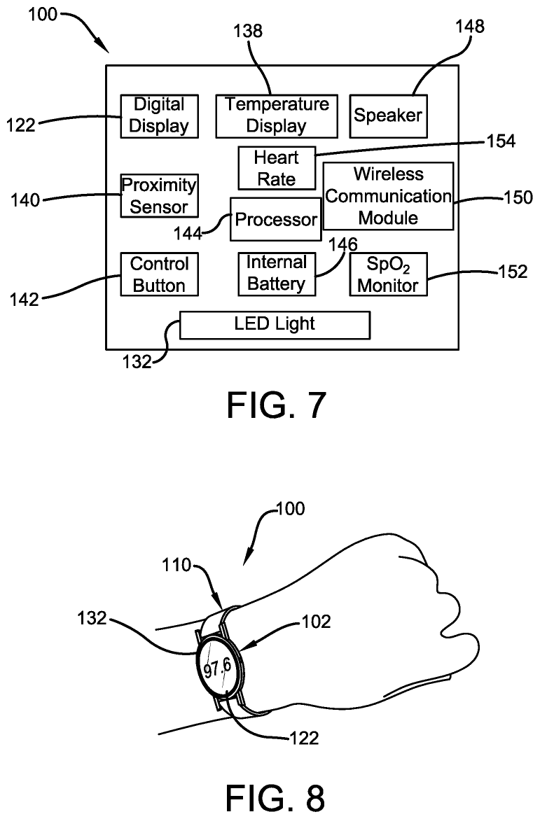

FIG. 7 illustrates a diagrammatic representation of exemplary components of the smart wearable device 100 of the present invention as per the disclosed structure. The smart wearable device 100 further comprises a user input 142. The user input 142 may be a dial attached to the case or a touch element within the digital display 122 for controlling components of the smart wearable device 100, such as units of measure, an/off, or reset. The smart wearable device 100 may further comprise a processor 144 and a wireless communications module 150.

The processor 144 is in communication with and configured to control and monitor operations of other components and to accept inputs from the user input 142. For example, the temperature sensing component 138 senses the temperature of skin of the wearer when the smart wearable device 100 is worn by the wearer on the wrist and the processor 144 executes an instruction to display the sensed temperature on the numerical indicator 124 of the digital display 120. The wireless communications module 150 is configured to communicate the measured health indicators with additional electronic platforms, such as a paired smartphone app. The smartphone app can be paired with the smart wearable device 100 using a wireless connection such as Bluetooth/Wi-Fi or any other similar wireless technology. The smart wearable device 100 is powered by an internal rechargeable battery 146.

The smart wearable device 100 may further comprise an oxygen saturation component 152. The oxygen saturation component 152 is integrated into the skin facing side 106 of the case 106 and is configured to measure oxygen saturation (SpO2) through the user's skin. The smart wearable device 100 may further comprise a pulse sensing component 154. The pulse sensing component 154 is integrated into the skin facing side 106 of the case 102 and is configured to measure a heart rate or the user. The plurality of health indicators may further comprise an oxygen saturation indicator 126 configured to display an oxygen saturation status, and a pulse indicator 128 to configured to display a heart rate status on the digital display. The lighting component 132 is also configured to illuminate in a first color when the any detected health indicator is within a normal range and illuminate in a second color when the detected health indicator is outside of the normal range. Similarly, the audible alert component 148 can indicate when any of the user's health indicators are detected outside of the normal temperature range.

As illustrated in FIG. 8, it should be noted that although the disclosure describes the smart wearable device 100 of the present invention as body health indicator measuring device, it should be appreciated that the smart wearable device can also work as a normal watch to display the time and date via the time/date indicator 130. The user input 142 may be used to switch the display 122 to show the current time, date or any other information such as temperature, heart rate, or SpO2 measurement. During this switch the lighting component 132 still illuminates in different colors, such as either green or red, to indicate the range of temperature of the wearer. The lighting component 132 remains illuminated while the smart wearable device 100 is worn by the wearer. Thus, the lighting component 132 also offers a way for people to travel in public and identify a fever symptom to prevent the spread of illness.

The smart wearable device 100 may be made available in different sizes and colors to accommodate user need and/or preference, and the exact size, measurement, construction and design specifications of the smart wearable device 100 of the present invention may vary upon manufacturing or the particular material that is used. The smart wearable device 100 may further comprise logos, indicia, trademarks, geometric patterns, customizable colors and fonts, embroidery and prints and/or images on its strap/case.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “temperature display wristwatch”, “temperature display wristwatch device”, “temperature watch”, “temperature wristwatch”, “wristwatch”, and “smartwatch” are interchangeable and refer to the smart wearable device 100 of the present invention.

Notwithstanding the forgoing, the smart wearable device 100 of the present invention can be of any suitable size and configuration as is known in the art without affecting the overall concept of the invention, provided that it accomplishes the above stated objectives. One of ordinary skill in the art will appreciate that the size, configuration and material of the smart wearable device 100 as shown in the FIGS. are for illustrative purposes only, and that many other sizes and shapes of the smart wearable device 100 are well within the scope of the present disclosure. Although the dimensions of the smart wearable device 100 are important design parameters for user convenience, the smart wearable device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A smart wearable device comprising:
a case;
a wristband attachable to the case;
a display component integrated into an outward facing side of the case; and
a temperature sensing component integrated into a skin facing side of the case; and
wherein the display is configured to visibly indicate a status of a user's skin temperature detected by the temperature sensing component.

2. The smart wearable device of claim 1, wherein the smart wearable device is a temperature sensing smart watch.

3. The smart wearable device of claim 1, wherein the display component comprises a digital display and a lighting component.

4. The smart wearable device of claim 3, wherein the digital display comprises a numerical temperature indicator.

5. The smart wearable device of claim 3, wherein the digital display comprises time indicator.

6. The smart wearable device of claim 3, wherein the lighting component is a LED strip surrounding a perimeter of the case.

7. The smart wearable device of claim 3, wherein the lighting component is configured to illuminate in a first color when the detected user's skin temperature is within a normal temperature range.

8. The smart wearable device of claim 7, wherein the lighting component is configured to illuminate in a second color when the detected user's skin temperature is outside of the normal temperature range.

9. The smart wearable device of claim 1, wherein the wristband comprises an illumination element for indicating when the detected user's skin temperature is outside of a normal temperature range.

10. The smart wearable device of claim 1 further comprising a proximity sensing component configured to activate the temperature sensing component.

11. The smart wearable device of claim 1 further comprising an audible alert component for indication when the detected user's skin temperature is outside of a normal temperature range.

12. The smart wearable device of claim 1 further comprising an oxygen saturation sensing component integrated into a skin facing side of the case.

13. A smart wearable device comprising:
a case;
a wristband attachable to the case;
a display component integrated into an outward facing side of the case; and
a plurality of health indicator sensing components integrated into a skin facing side of the case; and
wherein the display is configured to visibly indicate a status of a user's health indicators detected by the plurality of health indicator sensing components.

14. The smart wearable device of claim 13, wherein the plurality of health indicator sensing components comprise a temperature sensor.

15. The smart wearable device of claim 13, wherein the plurality of health indicator sensing components comprise an oxygen saturation sensor.

16. The smart wearable device of claim 13, wherein the plurality of health indicator sensing components comprise a pulse detecting sensor.

17. The smart wearable device of claim 13, wherein the display component comprises a digital display and a lighting component.

18. The smart wearable device of claim 17, wherein the lighting component is configured to illuminate in a first color when the detected user's health indicators are within a normal range and illuminate in a second color when at least one of the detected user's health indicators are outside of the normal range.

19. The smart wearable device of claim 13 further comprising an audible alert component for indication when any of the user's health indicators are detected outside of the normal temperature range.

20. A temperature sensing wearable device comprising:
a case;
a wristband attachable to the case;
a display component comprising a digital display and a lighting component integrated into an outward facing side of the case;
a plurality of health indicator sensing components integrated into a skin facing side of the case; and
a proximity sensing component configured to activate the plurality of health indicator sensing components; and
wherein the display is configured to visibly indicate a status of a user's health indicators detected by the plurality of health indicator sensing components.
